(19) United States
(12) Patent Application Publication
(10) Pub. No.: US 2006/0266368 A1
(43) Pub. Date: Nov. 30, 2006

(54) Pre-surgical safety, warning notification and/or safety device

(76) Inventor: James Aaron Heintz, Richboro, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 11/441,958

(22) Filed: May 26, 2006

Related U.S. Application Data
(63) Continuation-in-part of application No. 09/729,888, filed on Dec. 4, 2000, now abandoned.
(60) Provisional application No. 60/169,399, filed on Dec. 6, 1999.

(51) Int. Cl. A61F 13/00

(52) U.S. Cl. 128/846

(57) ABSTRACT

A method for providing a pre-surgical warning for use on a body of a patient having an involved body segment and a plurality of uninvolved body segments includes providing associated first, second, third and fourth visually perceptible indicia, disposing the first, second, third and fourth upon first, second, third and fourth carriers, respectively, determining the involved body segment and at least one uninvolved body segment, removing the first, second, third and fourth indicia from the first, second and third carriers, disposing the first and second indicia in the vicinity of the determined involved body segment and in the vicinity of the determined at least one uninvolved body segment, respectively, to provide a labeled involved body segment and a labeled uninvolved body segment and disposing the third and fourth indicia at at least one location remote from the patient to indicate that the first and second indicia are disposed upon the patient.

### CROSS-REFERENCE TO RELATED APPLICATIONS

This application is a continuation-in-part of U.S. patent application Ser. No. 09/729,888, filed on Dec. 4, 2000, which is based upon U.S. Provisional Patent Application Ser. No. 60/169,399, filed on Dec. 6, 1999, entitled PRE-SURGICAL SAFETY, WARNING NOTIFICATION AND/OR SAFETY DEVICE.

### BACKGROUND OF THE INVENTION

1. FIELD OF INVENTION

This invention relates to the field of devices for providing medical alert information to healthcare workers.

2. DESCRIPTION OF RELATED ART

Surgical mistakes are tragic for the patient and costs hundreds of millions of dollars each year in lawsuits and damage awards against doctors, hospitals and/or other health care providers. Media attention has exposed this disturbing trend, but there has been no practical solution until the advent of the present invention.

The invention is an instantly recognizable method of warning the surgical health care provider(s) that they are not at the intended surgical site. It is an opportunity to improve patient care and reduce risk, and should be a basic step in the surgical preparation process. Why should one more patient suffer the dire consequences of having the wrong limb amputated or the wrong organ removed? Why should insurance companies and consumers pay one more huge malpractice award when use of this product could so easily make such losses unnecessary?

The invention will benefit surgical patients, insurers, consumers and the health care community.

This invention pertains to an instantly recognizable method of pre-surgically warning the surgical health care provider(s) whether they are at the intended surgical site.

It is a novel method of providing a pre-surgical safety warning, notification, and/or alert, intended to help avoid surgical procedures from being accidentally performed on patients' unintended limbs, tissue and/or other body parts.

The American Hospital Association reports that in 1998 there were 25 million hospital surgical procedures for a population of 280 million (9% of population). The procedures and practices that are currently in place to avoid unintended surgeries are insufficient and ineffective.

Patient comfort, protection of body temperature and the identification of the patient, surgeon and surgical site is addressed in U.S. Pat. No. 4,947,867 by Keeton. The Keeton patent is primarily concerned with the comfort of the patent by providing the patient with a disposable surgical gown of sufficient material to retain body temperature during surgery as well being able to be cut away at the surgical site.

While the above cited art is useful for its intended purpose of being disposable and keeping the identified patient at a comfortable temperature, it does nothing to pre-surgically warn surgical health care providers whether they are at the intended surgical site.

The within invention is distinguishable from prior art in that the primary purpose and embodiment of the invention is as a pre-surgical safety, warning, notification and/or alerting device, intended to alert surgical health care providers whether they are the intended surgical site. An object of this invention is to provide a novel pre-surgical method of providing safety, warning, notification and/or alerting device, intended to help avoid accidental surgical procedures from being performed on patients' unintended limbs, tissue and/or other body parts.

All references cited herein are incorporated herein by reference in their entireties.

### BRIEF SUMMARY OF THE INVENTION

A method for providing a pre-surgical warning for use on a body of a patient having an involved body segment to be involved in a surgical procedure and a plurality of uninvolved body segments which are not to be involved in the surgical procedure, including providing associated first, second, third and fourth visually perceptible indicia, disposing the first, second, third and fourth upon first, second, third and fourth carriers, respectively, determining the involved body segment and at least one uninvolved body segment, removing the first, second, third and fourth indicia from the first, second, third and fourth carriers, disposing the first and second indicia in the vicinity of the determined involved body segment and in the vicinity of the determined at least one uninvolved body segment, respectively, to provide a labeled involved body segment and a labeled uninvolved body segment and disposing the third and fourth indicia at at least one location remote from the patient to indicate that the first and second indicia are disposed upon the patient. The at least one remote location is a patient chart. The first carrier is the second carrier and/or third carrier. The third indicia is disposed on at least one of the first and second carriers. At least one of the first, second, third and fourth indicia is peelably from the at least one of the first and second carriers. The indicia are numerical sequential indicia and sequential indicia, tattoo indicia and bar code indicia. The first indicia is the third indicia and the second indicia is the fourth indicia. The first indicia is the second, third and fourth indicia.

A method is disclosed for providing a pre-surgical warning for use on a body of a patient having an involved body segment to be involved in a surgical procedure and a plurality of uninvolved body segments which are not to be involved in the surgical procedure, including the steps of providing corresponding first and second associated visually perceptible indicia and disposing the first visually perceptible indicia upon a surface of a label. The involved body segment of the patient is determined and the label is disposed on the involved body segment of the patient to provide a labelled involved body segment for indicating that the surgical procedure is to be performed on the labelled involved body segment. The second visually perceptible indicia is disposed at a location remote from the patient to indicate that the first visually perceptible indicia is disposed on the body segment of the patient. The remote location can be a patient chart. The label can be disposed on a first carrier. The second visually perceptible indicia can be disposed on a second carrier. The first carrier can be the same as the second carrier. At least one of the visually perceptible indicia can be peelably disposed on a carrier of the first and second carriers or both of the first and second visually perceptible indicia can be peelably disposed upon a carrier of the first and second carriers. The indicia can be sequential numerical indicia.

A pre-surgical alerting device for use on a body having an involved body segment to be involved in a surgical procedure includes a first warning label for disposing the first warning label on the involved body segment to provide a labelled involved body segment and a visually perceivable indicator disposed on the first warning label for indicating that the surgical procedure is to be performed on the involved body segment. A second warning label corresponding to the first warning label indicates that the first warning label is disposed on the involved body segment. First and second identifying indicia are disposed on the first and second warning labels, respectively. A first carrier detachably bears the first warning label and a second carrier detachably bears the second warning label. The first carrier can be the same as the second carrier. The first and second identifying indicia can be corresponding identifying indicia. The identifying indicia can be sequential numerical indicia. At least one of the first and second warning labels is peelably attached to a carrier of the first and second carriers and both the first warning label and the second warning label can be peelably attached to the carrier of the first and second carrier. The second warning label can be disposed on a patient chart.

### BRIEF DESCRIPTION OF SEVERAL VIEWS OF THE DRAWINGS

The invention will be described in conjunction with the following drawings in which like reference numerals designate like elements and wherein:

FIG. 1 is a front view of a pre-surgical alerting device, according to the present invention.

FIG. 2 is a view of both sides of a pre-surgical alerting device, according to the present invention.

FIG. 3 is a front view of another pre-surgical alerting device, according to the present invention.

FIG. 4 is a front view of a pre-surgical alerting and notifying device, according to the present invention.

FIG. 5 is a front view of a pre-surgical alerting and notifying device, according to the present invention.

### DETAILED DESCRIPTION OF THE INVENTION

The present invention concerns a novel method of providing a pre-surgical safety, warning, notification, and/or alerting device to help avoid surgical procedures from being accidentally performed on patients' unintended limbs, tissue and/or other body parts.

In the following description, numerous specific details are set forth in order to provide thorough understanding of the present invention. It will be obvious, however, to one skilled in the art that the present invention may be practiced without these specific details. Some well-known methods and structures have not been set forth in order not to unnecessarily obscure the description of the present invention.

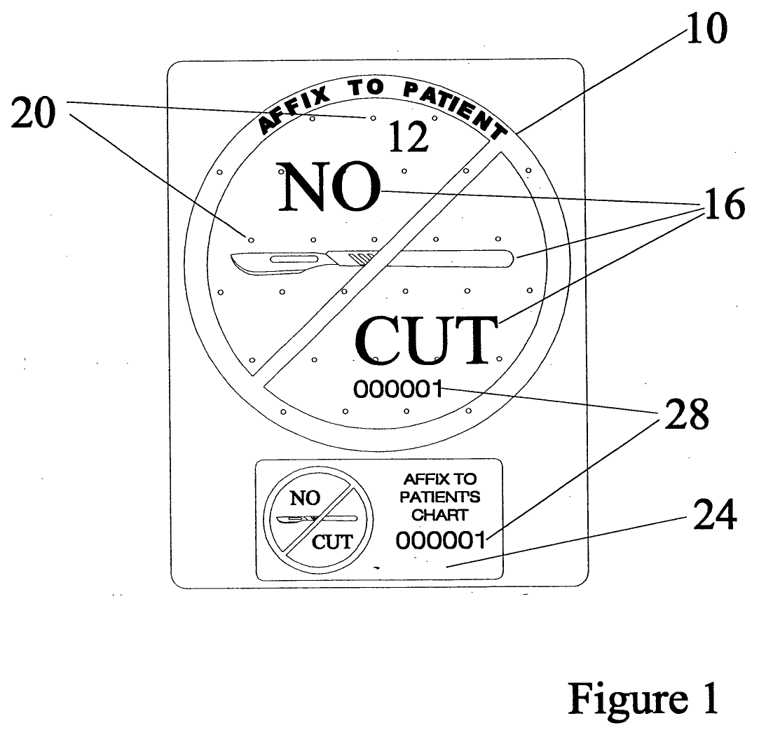

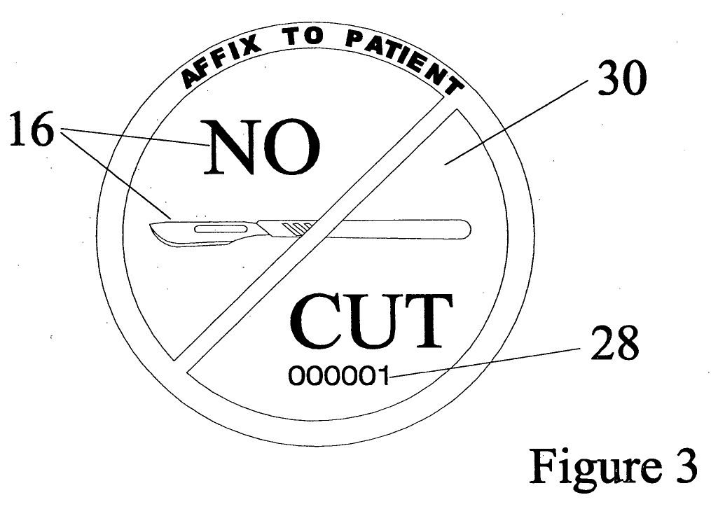

The preferred embodiment of the invention includes a vinyl, PVC, cellulose, woven filament, fabric, label or other material strip 10 of various sizes (e.g. 2 inches by 3 inches) and/or shapes (rectangle, square, round or other) in various color combinations (red, white, etc.). As shown in FIG. 1, the label or strip 10 may be constructed of material with perforations 20 to allow oxygen to diffuse to the underlying skin. Such a strip 10 may be similar in construction to bandage wound care products without the wound pad. Alternatively, as shown in FIG. 3, a skin-penetrative applique 30 such as a temporary tattoo may be attached to a patient as the vehicle for alerting health care providers to a site which is not intended for a medical procedure. The strip 10 is intended to remain in place for between 24 to 48 hours.

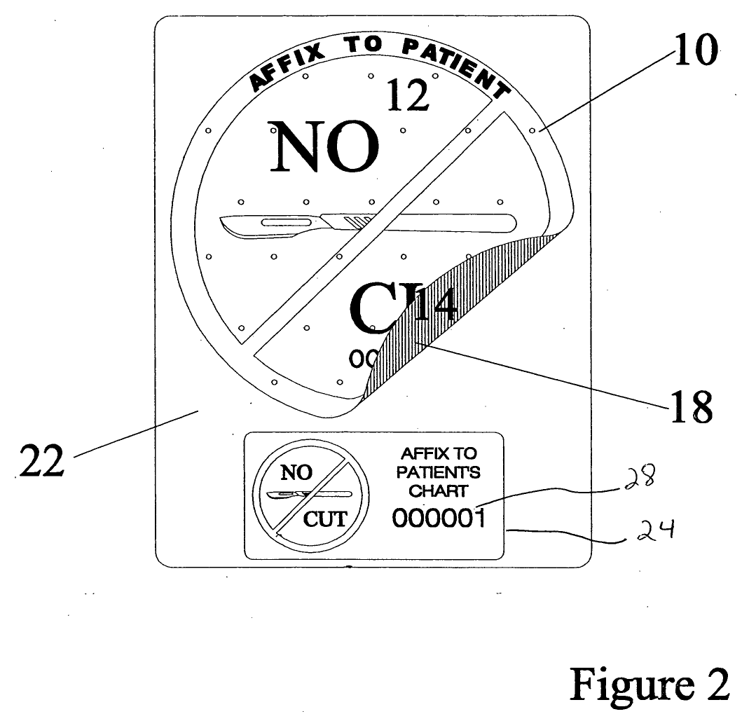

Words, pictorial images, and/or other visually acceptable warning message(s) 16 are printed on the superior side 12 of the strip 10. As best shown in FIG. 2, the inferior side 14 of the strip 10 is coated with adhesive 18, similar to that customarily used on bandage wound care products. In this manner, the strip 10 can be affixed to the skin of a patient prior to surgery or other medical procedure. Non-stick peelable backing 22 or carrier 22 is affixed to the inferior side 14 of the strip 10 and is intended to be removed prior to use. The invention is/may be packaged in a sterile sleeve envelope or an a continuous roll (not shown).

The strip 10 may be affixed to the patient pre-surgically by the patient, physician, and/or other health care provider. The invention may beneficially include a companion label 24 which can be a miniature version of the strip 10. The companion label 24 is intended to be affixed to a location remote from the patient such as the patient's medical chart (not shown) to document that the alerting strip 10 has been utilized and has been affixed to pertinent or appropriate area(s) of the patient in order to warn the surgical health care provider(s) not to cut at an intended surgical site. As shown in FIG. 1, visually perceptible indicia 28 on both the alerting strip 10 and the corresponding companion label 24 establish that a particular strip 10 is related to or associated with the companion label 24 with the corresponding indicia 28. The associated indicia 28 can be numerical indicia. Additionally, indicia 28 can be sequential numerical indicia in order to facilitate record keeping and the review of medical records related to the surgery. The corresponding companion label 24 and the strip 10 can preferably be provided on a common carrier, although this is not necessary and they can be provided separately.

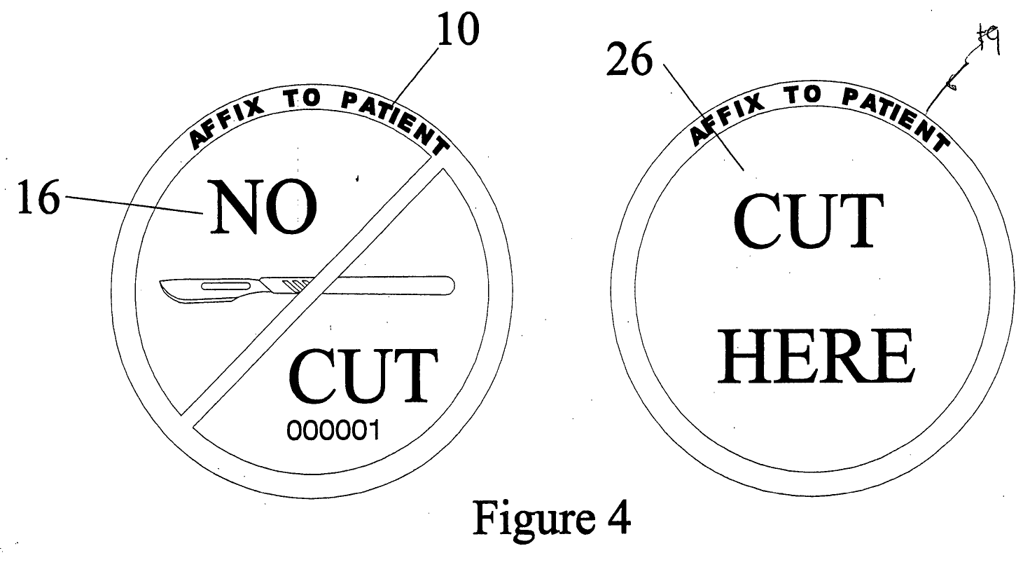

As shown in FIG. 4, a system comprising an alerting strip 10 and a corresponding strip 26 may be used, to establish with certainty the appropriate location for a medical procedure. When the system shown in FIG. 4 is used, the alerting strip 10 is attached to an area of the patient's body which should not be effected by a surgical procedure. The corresponding strip 26 can be purposely attached to the area of the patient's body which should be effected by the surgical procedure, and provides notice of the proper surgical site by a visual indicator. Either label 24 or strip 10 can be used by itself to indicate the site to cut or not to cut respectively. The visual indicator of the corresponding strip 26 can be as simple as the words “CUT HERE”, although it will be understood that other indicators can also be used.

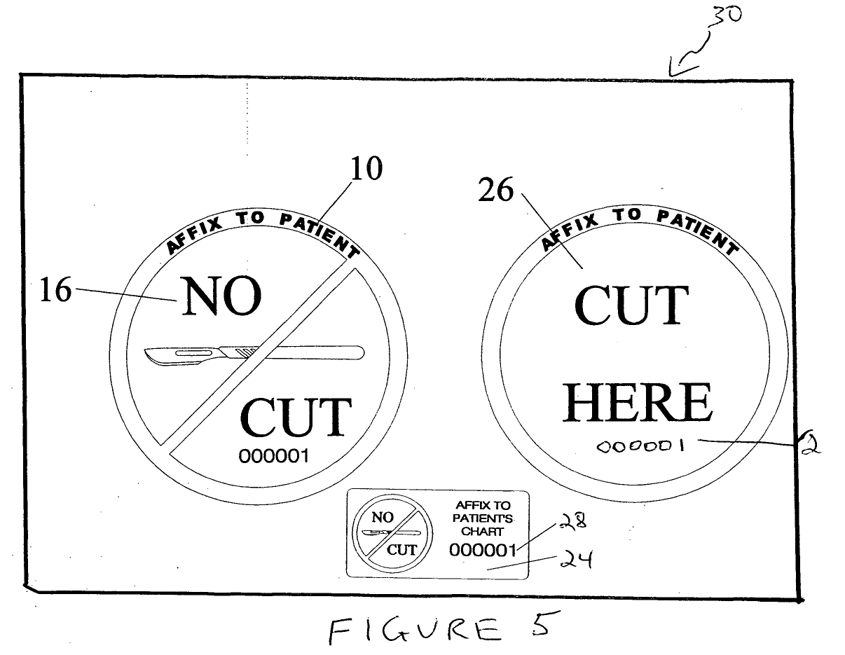

As shown in FIG. 5, a system including at least one carrier 30 can be provided according to the system and method of the invention. A no cut strip 10 and an cut here strip 26 can be associated with respective carriers 30. Additionally, the no cut strip 10 and the cut here strip 26 can both be associated with a single carrier 30. Furthermore, one or more companion labels 24 or companion indicators 24 can be associated with the single carrier 30 or with plural carriers 30. The companion labels can be removed from the carriers 30 and disposed on different locations, such as different charts, different pieces of paper work of other types and different objects, such as surgical objects.

One or more of the no cut strip 10, the cut here strip 26 and the companion labels 24 can be provided with indicia 28. An indicia associated with a strip 10, a strip 26 or one or more of the companion labels 24 can be adapted to correspond to other indicia in order to indicate an association there between. In this manner, when an indicator such as a strip 10, a strip 26 or a companion label 24 is removed from the proximity of a remaining strip 10, strip 26 or companion label 24, a determination can be made which remaining strip 10, strip 26 or companion label 24 are associated with the removed one.

In addition to an embodiment of the invention wherein the carrier 30 can carry a companion label 24 having an indicia 28 corresponding to the indicia of both the no cut strip 10 and the cut here strip 26, an alternate embodiment of the invention the carrier 30 can carry two or more companion labels 24. The two or more companion labels 24 can have corresponding indicia or differing indicia. An indicia of one of the companion labels 24 or companion indicators 24 can correspond to the indicia of the no cut strip 10. The indicia of another companion indicator 24 can correspond to the indicia of the no cut strip 10.

The indicia 28 can be numerical indicia. Additionally, the indicia 28 can be any other kind of indicia known to those skilled in the art for indicating associations there between, including bar codes, RFID, magnetic tape, etc. Furthermore, the indicia 28 can be colors, images, text, etc. Additionally, the no cut strip 10 and the strip 26 can each be used with a respective companion label 24 independently of each other in the manner previously described, in order to label only the involved body segment or only the uninvolved body segment. In an alternate embodiment of the invention the no cut strip 10, the cut here strip 26 and the companion indicator 24 can be on separate carriers.

In one embodiment, wherein the no cut strip 10 and the cut here strip 26 can be associated with each other by the indicia 28, the strips 10, 26 can be used simultaneously on a patient, to explicitly indicate both the involved body segment and the uninvolved body segment of the patient. The indicators of the involved and uninvolved body segments, and/or the associated companion indicator or indicators, can be disposed on the same carrier or on plural carriers. A no cut strip 10 and a cut here strip 26 that are not associated with each other by an indicia 28 can also be used simultaneously on a patient to explicitly indicate both the involved and uninvolved body segments. A no cut strip 10 and a cut here strip 26 can thus have companion labels 24 that are respectively associated only with themselves.

Furthermore, additional indicia can be provided on the indicators, such as strips and companion labels, wherein: (1) a single association is provided between the strips and the companion labels, and (2) further associations are provided between each strip and its respective companion label.

The foregoing constitutes the best mode known by the applicant for carrying out this invention by one of ordinary skill in the art. However, the specific embodiments disclosed are selected for the purpose of illustrating the principles of the system and method of the invention and are not limiting in scope. To the contrary, it is recognized that one of ordinary skill in the art, given this teaching, may make any variations in the structure, compositions or methods disclosed without departing from the spirit and scope of this invention as set forth in the claims. The scope of the invention is thus defined by the following claims including the protection offered by the doctrine of equivalents.

While the invention has been described in detail and with reference to specific examples thereof, it will be apparent to those skilled in the art that various changes and modifications can be made therein without departing from the spirit and scope thereof.

1. A method for providing a pre-surgical warning for use on a body of a patient having an involved body segment to be involved in a surgical procedure and a plurality of uninvolved body segments which are not to be involved in said surgical procedure, comprising:
a. providing associated first, second, third and fourth visually perceptible indicia;
b. disposing said first, second, third and fourth upon first, second, third and fourth carriers, respectively;
c. determining said involved body segment and at least one uninvolved body segment;
d. removing said first, second, third and fourth indicia from said first, second, third and fourth carriers;
e. disposing said first and second indicia, in the vicinity of said determined involved body segment and in the vicinity of said determined at least one uninvolved body segment, respectively, to provide a labeled involved body segment and a labeled uninvolved body segment; and
e. disposing said third and fourth indicia at least one location remote from said patient to indicate that said first and second indicia are disposed upon said patient.

2. The method for providing a pre-surgical warning of claim 1, wherein said at least one remote location comprises a patient chart.

3. The method for providing a pre-surgical warning of claim 1, wherein said first carrier comprises said second carrier.

4. The method for providing a pre-surgical warning of claim 3, wherein said first carrier comprises said third carrier.

5. The method for providing a pre-surgical warning of claim 1, wherein said third indicia is disposed on at least one of said first and second carriers.

6. The method for providing a pre-surgical warning of claim 5, further comprising peelably removing at least one of said first, second, third and fourth indicia from said at least one of said first, second, third and fourth carriers.

7. The method for providing a pre-surgical warning of claim 6, further comprising peelably removing at least two of said first, second, third and fourth indicia from at least one of said first, second, third and fourth carriers.

8. The method for providing a pre-surgical warning of claim 1, further comprising providing numerical indicia.

9. The method for providing a pre-surgical warning of claim 8, further comprising providing sequential indicia.

10. The method for providing a pre-surgical warning of claim 1, further comprising providing tattoo indicia.

11. The method for providing a pre-surgical warning of claim 1, further comprising providing bar code indicia.

12. The method for providing a pre-surgical warning of claim 1, wherein said first indicia comprises said third indicia.

13. The method for providing a pre-surgical warning of claim 12, wherein said second indicia comprises said fourth indicia.

14. The method for providing a pre-surgical warning of claim 1, wherein said first indicia comprises said second, third and fourth indicia.

15. A pre-surgical warning system for use on a body of a patient having an involved body segment to be involved in a surgical procedure and a plurality of uninvolved body segments which are not to be involved in said surgical procedure, comprising:
a. first and second associated visually perceptible indicia respectively disposed in the vicinity of said involved body segment and in the vicinity of said at least one uninvolved body segment to provide a labeled involved body segment and a labeled uninvolved body segment; and
e. a third and fourth indicia visually perceptible indicia associated with said first and second associated indicia disposed at at least one location remote from said patient to indicate that said first and second indicia are disposed upon said patient.

16. The system for providing a pre-surgical warning of claim 15, wherein said remote location comprises a patient chart.

17. The system for providing a pre-surgical warning of claim 15, further comprising a carrier for bearing said first, second, third and fourth indicia prior to disposing said first and second indicia upon said patient.

18. The method for providing a pre-surgical warning of claim 17, wherein at least one of said first, second, third and fourth indicia is peelably attached to said carrier.

19. The system for providing a pre-surgical warning of claim 15, wherein said indicia comprise numerical indicia.

20. The system for providing a pre-surgical warning of claim 19, further comprising sequential indicia.
